(19) United States
(12) Patent Application Publication
(10) Pub. No.: US 2006/0074307 A1
(43) Pub. Date: Apr. 6, 2006

(54) Body cavity diagnostic system

(76) Inventors: Tatsuo Igarashi, Chiba (JP); Yoichi Miyake, Chiba (JP); Toshiya Nakaguchi, Chiba (JP); Harufumi Makino, Chiba (JP); Hiroshi Fujita, Saitama (JP)

Correspondence Address: YOUNG & THOMPSON, 745 SOUTH 23RD STREET, 2ND FLOOR, ARLINGTON, VA, 22202, US

(21) Appl. No.: 11/125,202

(22) Filed: May 10, 2005

(30) Foreign Application Priority Data
May 10, 2004 (JP) 2004-139736
May 12, 2004 (JP) 2004-142604

(51) Int. Cl. A61M 25/00

(52) U.S. Cl. 600/434

(57) ABSTRACT

A body cavity diagnostic system includes a medical procedure instrument for practicing a surgical procedure on an affected part within a body cavity of a patient, a body cavity diagnostic unit for optically capturing an image of an interior of the body cavity, the body cavity diagnostic unit being fixedly put in an incision formed in a body wall of the patient; and a monitor unit for magnifying and displaying a desired part of the image on a monitor screen. Either one of the medical procedure instrument and the cannula is provided with a position representation mark on its distal end. A part of the image detected the mark detection element is trimmed and magnified so as to be displayed in a center area of the monitor screen.

### BACKGROUND OF THE INVENTION

1. Field of the Invention

The present invention relates to a body cavity observation system and, more particularly, to a body cavity observation system for performing invasive surgery or invasive surgical procedures on a human or animal patient with visual observation of body channels, cavities, spaces and internal organs on a monitor.

2. Description of Related Art

Laparoscopic devices, one of medical endoscopes enabling visual observation or examination of body channels, cavities, spaces and internal organs of a human or animal patient, are used to perform a variety of surgical procedures seeing an image of an affected part of the body, for example adhesion, ovarian tumors, uterus myomas, on a monitor. Such a laparoscope is inserted through the skin to access a body cavity through a trocar sleeve or tube. In order to penetrate the skin, the distal end of the trocar sleeve is placed against the skin and a trocar is inserted through the trocar sleeve. By pressing against the proximal end of the trocar, the point of the trocar is forced through the skin to produce an incision until it enters the body cavity. At this time, the trocar sleeve is inserted through the incision made by the trocar. Then, the trocar is withdrawn, leaving the trocar sleeve as an access way into the body cavity. During a surgical procedure, the laparoscope is fixedly held by a movable holder fixed to a stationary part of a body wall of a patient. Such a laparoscopic diagnostic and procedure device is disclosed in, for example, Japanese Unexamined Patent Publication No. 2003-265402.

In practicing the laparoscopic surgeries or procedures, it is general to produce three to four incisions in a body wall of a patient for insertion of a plurality of medical procedure instruments through trocars. One or two operating surgeons operate the medical procedure instruments viewing an image of the body cavity captured by a laparoscope on a monitor. In addition to the operating surgeons, one or two assistant surgeons operate the laparoscope and provide general clinical operation.

The prior art body cavity diagnostic system has an essential need for four to five operating and assistant surgeons, and, what is worse still, they are hampered by a plurality of cables such as a light guide cable extending from a light source and a signal output cable extending from a charge coupled device (CCD) image sensor around a surgical table. Therefore, it is waited for a body cavity diagnostic system manipulated by a few surgeons.

### SUMMARY OF THE INVENTION

It is therefore an object of the present invention to provide a body cavity diagnostic system manipulated y a few surgeons and has a decreased number of cables extending around a surgical table.

The foregoing object of the present invention is accomplished by a body cavity diagnostic system comprising a body cavity diagnostic unit for optically capturing an image of an interior of the body cavity and transmitting image signals of the image toward image receiving means for visual diagnosis, the body cavity diagnostic unit being fixedly put in an incision formed in a body wall of the patient. The body cavity diagnostic unit comprises illumination means for illuminating an interior of the body cavity, an image pickup optical system including solid state image sensing means for converting an image formed thereon into image signals, signal transmitting means for transmitting the image signals by air, and a power source for supplying electric power to the illumination means, the solid state image sensing means and the signal transmitting means.

According to another preferred embodiment of the invention, the body cavity diagnostic system comprises a medical procedure instrument for practicing a surgical procedure on an affected part within a body cavity of a patient; the medical procedure instrument being inserted into the body cavity through a cannula as an access way, a body cavity diagnostic unit for optically capturing an image of an interior of the body cavity, the body cavity diagnostic unit being fixedly put in an incision formed in a body wall of the patient, and a monitor unit for magnifying and displaying a desired part of the image on a monitor screen.

The body cavity diagnostic and procedure preferably comprises a position representation mark formed on a distal end of at least one of the medical procedure instrument and the cannula The monitor unit trims the image so as to center the position representation mark in the desired area of the magnified image displayed on the monitor screen. This image processing makes it easy to magnify and display a real affected part in a body cavity without the assistance of someone else.

The body cavity diagnostic system eliminates the necessity of an operating surgeon who operates a laparoscope viewing an image of a body cavity of a patient on a monitor screen. The body cavity diagnostic unit that includes the illumination means therein driven by a built-in battery and transmits image signal by air eliminates the necessity of a light guide cable and a signal output cable which extend around a surgical table.

An optical system of the body cavity diagnostic unit generally has a wide angle of view because it is fixedly put in an incision formed in a body wall of the patient. However, if displaying a complete image captured by the wide angle optical system, it is difficult to make an examination of a real affected part in the body cavity. This inexpediency is eliminated by trimming and magnifying a part of the image detected by the mark detection means for detecting a mark on the medical procedure instrument so as to be displayed in a center area of the monitor screen even though the image is captured by the wide angle optical system. Therefore, the operating surgeon can manipulate the medical procedure instrument without operating the body cavity diagnostic unit fixedly put in an incision formed in a body wall of a patient

The body cavity diagnostic system is preferably provided with the body cavity diagnostic unit having an interchangeable optical system. The solid state image sensing means of the interchangeable optical system and the signal transmitting means of the body cavity diagnostic unit are electrically coupled through connectors, so that image signals from the solid state image sensing means are transmitted to the signal receiver means on the monitor side by air.

The illumination means may comprise one or more light emitting diodes that are significantly smaller in size than illumination bulbs. This provides a small size of body cavity diagnostic unit small in overall size, and saves electric power besides. It is preferred to use a light emitting diode different in color from a position representation mark for accurate detection.

It is more preferred that the body cavity diagnostic system further comprising image signal receiving means for receiving the image signals transmitted by air from the signal transmitting means, monitor means for displaying an visual image based on the image signals received by the signal receiving means, and a medical procedure instrument for practicing a surgical procedure on an affected part within the body cavity; the medical procedure instrument being inserted into the body cavity through a cannula as an access way.

The body cavity diagnostic unit may comprise a laparoscopic diagnostic system or a thoracoscopic diagnostic system.

### BRIEF DESCRIPTION OF THE DRAWINGS

The foregoing and other objects and features of the present invention will be clearly understood from the following detailed description when reading with reference to the accompanying drawings, wherein the same reference signs have been used to denote same or similar parts throughout the drawings, and in which:

FIG. 1 is a schematic illustration showing a body cavity diagnostic system having a body cavity diagnostic unit with an image capturing optical system built therein according to an embodiment of the present invention;

FIG. 2 is an explanatory view showing a clinical operation using the body cavity diagnostic system;

FIG. 3 is a block diagram of a system configuration of the body cavity diagnostic system FIG. 4 is a schematic illustration showing visual axis tracking system;

FIG. 5 is an explanatory view showing a clinical operation using a body cavity diagnostic system according to another embodiment of the present invention; and

FIG. 6 is an explanatory view showing a clinical operation using a laparoscope as the body cavity diagnostic system.

### DETAILED DESCRIPTION OF THE PREFERRED EMBODIMENTS

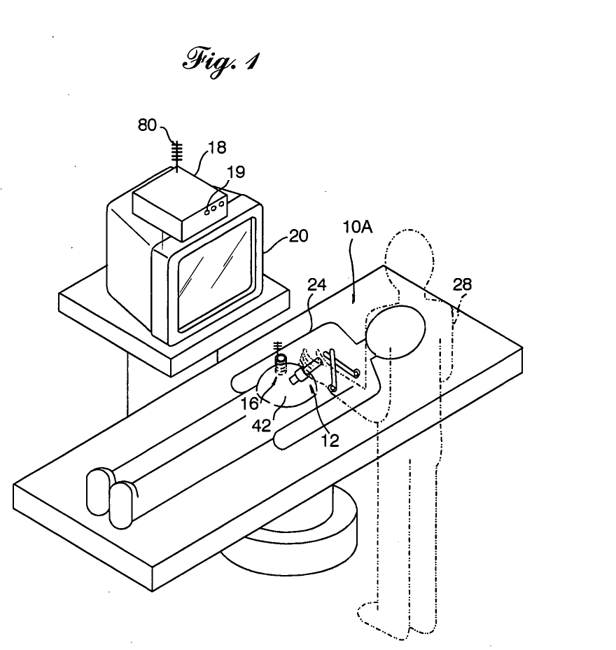

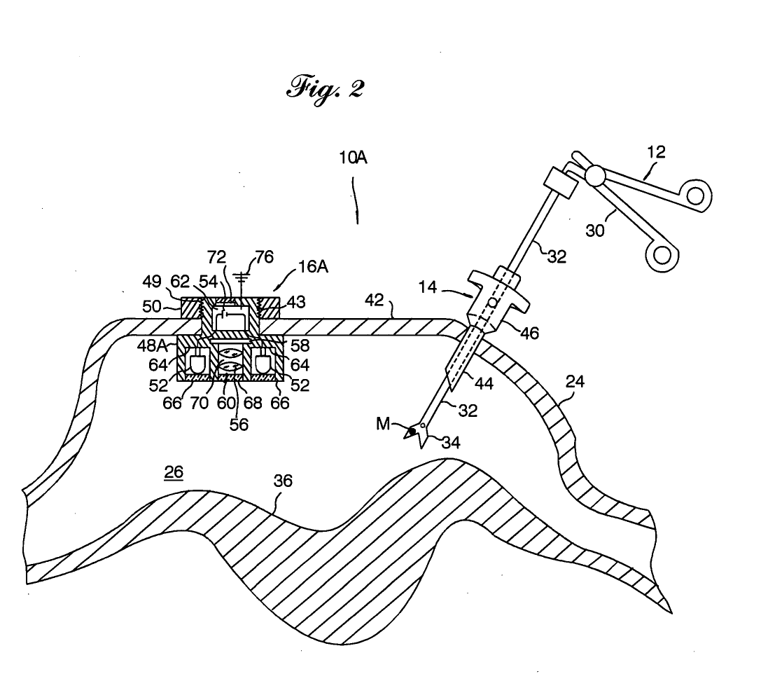

Referring to the accompanying drawings in detail, and in particular, to FIGS. 1 and 2 showing a body cavity diagnostic system 10A according to one embodiment of the present invention, the body cavity diagnostic system 10A comprises a surgical instrument 12, a hollow trocar 14, a body cavity diagnostic unit 16A, an image processing unit 18, a monitor 20 and so on. The surgical instrument 12, that is used for practicing a laparoscopic surgical procedure on an affected part 26 within a body cavity, such as an abdominal cavity 26, of a human or animal patient 24 lying down on a surgical bed 22, comprises an operating portion 30 though which a surgeon 28 operates the surgical instrument 12, a longitudinal shaft 32 and a pair of forceps 34. The surgical instrument 12 is placed in the hollow trocar 14 inserted through an abdominal wall 42, namely layers of skin and human flesh, of the patient 24 and left in the abdominal wall 42 as an access way into the abdominal cavity 26 so that the forceps 34 protrude from the distal end of the hollow trocar 14. The hollow trocar 14 is made up of a metal tube 44 having a sharp point at a distal end for easy penetration and a gripper tube 46 at a proximal end which the operating surgeon 28 grips. In order to penetrate the skin, the operating surgeon 28 grips the gripper tube 46 of the trocar 14 and places and presses the sharp point against the skin of the abdominal wall 42 to force the sharp point through the skin until it enters layers of the skin and flesh of the abdominal wall 42. At this time, the metal tube 44 of the hollow trocar 14 is inserted through a perforation made by the sharp point and, then, the hollow trocar 14 is left in the abdominal wall 42 as an access way into the abdominal cavity 26.

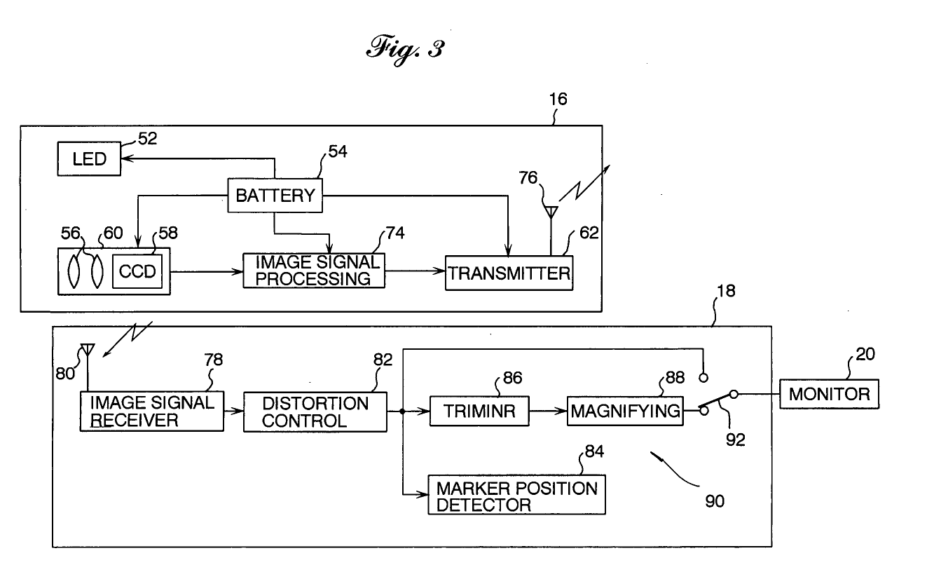

The body cavity diagnostic unit 16A is fixedly put in an incision 43 formed in the abdominal wall 42 near the perforation for penetration of the hollow trocar 14. This body cavity diagnostic unit 16A has a cylindrical unit housing 48 with a threaded cylindrical collar 49 and a collect ramp 50 tightened to the threaded cylindrical collar 49 to anchor the body cavity diagnostic unit 16A to the abdominal wall 42 therebetween, so as thereby to hold the body cavity diagnostic unit 16A in the incision 43 of the abdominal wall 42. In the interior of the unit housing 48 there are provided a plurality of light emitting diodes (LEDs) 52 received within circular compartment 64 formed in the unit housing 48, a small, lightweight power source such as a button type battery 54, an image pickup optical system 60 comprising an optical lens system 56 received within a cylindrical center compartment 70 and a solid state image sensing device such as a charge coupled device (CCD) image sensor 58 disposed behind the optical lens system 56, a transmitter 62 for sending out image signals by air, and an image signal processor 74 (see FIG. 3) for processing image signals from CCD image sensor 58. LEDs 52 are arranged on a circle having a center on an optical axis of the optical lens system 56 at regular angular intervals. The unit housing 48 at its distal end is provided with a transparent diffusion plate 66 for air-tightly covering LEDs 52 and a transparent plate 68 for air-tightly covering the optical lens system 56. The button type battery 54 is removably disposed on a bottom of the threaded cylindrical collar 49. The unit housing 48 at its proximal end is further provided with a cap 70 detachably thereto. The cap 70 is detached for replacement of the button type battery 54 with a fresh one. The battery 54 supplies electric power to LEDs 54, CCD image sensor 58 and the transmitter 62 as well as an image signal processor 74.

The body cavity diagnostic unit 16A thus constituted illuminates an object 36, namely an affected part within the abdominal cavity 26 with light emanating from LEDs 52 and forms an optical image on CCD image sensor 58 through the optical lens system 56. Image signals into which CCD image sensor 58 converts the optical image formed thereon are processed in the image signal processor 74 and then sent to the image signal processing unit 18 shown in FIG. 1 from the transmitter 62 through an antenna 76. Therefore, the body cavity diagnostic unit 16A has the function of lighting a body cavity, imaging the body cavity and transmitting the image of body cavity.

FIG. 3 is a block diagram showing the body cavity diagnostic unit 16A and the image signal processing unit 18 between which signal communication is made. The image signal processing unit 18 comprises an image signal receiver 78 provided with a receiving antenna 80, distortion control means 82 and marker tracking means (image area selection and magnification means) 90 including mark detection means 84, trimming means 86 and magnifying means 88. The image signals output from the image signal processing unit 18 through the transmitting antenna 76 are received by the image signal receiver 78 through a receiving antenna 80 and then transmitted to the distortion control means 82 for correction of image distortion occurring due to the optical property of the optical lens system 56 of the image pickup optical system 60. After the distortion correction, the image signals are further transmitted to the marker tracking means 90.

Detailed description of the marker tracking means 90 will be given below. For the reason that the body cavity diagnostic unit 16A is fixedly put in an incision 43 formed in the abdominal wall 42, it is desired that the image pickup optical system 60 has an angle of view sufficiently large enough to take a survey of the whole area of the abdominal cavity 26. However, in the case where the monitor 20 displays an image of the whole area of the abdominal cavity 26 optically captured by the body cavity diagnostic unit 16A through the image pickup optical system 60 having a large angle of view such as, for example, 170°, the image on the monitor is too small to conduct real visual examination on a part of the abdominal cavity 26 near the distal end of the surgical instrument 12 and/or the hollow trocar 14. In order to overcome this problem, the forceps 34 of the surgical instrument 12 is provided with a position representation mark M distinctively colored, for example, blue that is visually perceptible differently from a body cavity color. During image processing in the marker tracking means 90, the mark detection means 84 distinguishes the position representation mark M on the forceps 34. Then, the trimming means 86 rims the image so as to center the position representation mark M in a predetermined size of frame. The magnifying means 88 magnifies the specific size of image at a desired magnification so as thereby to fill up a screen of the monitor 20. The image processing unit 18 has a magnification control knob 19 for providing any desired magnification. In this way, the operating surgeon 28 is enabled to examine a real affected part of the abdominal cavity 26 near the forceps 23 of the surgical instrument 12 at a center on a macro image displayed on the monitor 20. That is to say, the marker tracking means 90 enables the operating surgeon 28 to concentrate on a surgical procedure without operating the body cavity diagnostic unit 16A fixedly put in an incision 43 formed in the abdominal wall 42 for seeking for the real affected part of the abdominal cavity 26 and needs lessens the need for assistant surgeons. Since the marker tracking means 90 always tracks the position representation mark M so as to center it on screen of the monitor 20, the operating surgeon and his or her assistant surgeons can examine an magnified image of a real affected part of the abdominal cavity 26 near the forceps 23 of the surgical instrument 12 even upon an occurrence of a positional change of the position representation mark M on the forceps 23 resulting operation of the surgical instrument 12 or the hollow trocar 14.

The image processing unit 18 is provided with a changing-over switch 92 for connecting the monitor 20 selectively to the distortion control means 82 and the magnifying means 88 for displaying selectively an image only after distortion correction on the monitor 20and an magnified image including the position representation mark M at a center on the monitor 20, respectively. When selecting the connecting of the monitor 20 to the distortion control means 82, an original image of the area of the whole cavity 26 optically captured by the body cavity diagnostic unit 16A is displayed and examined on the monitor 20. The selection of connection of the monitor 20 to the distortion control means 82 enables the operating surgeon and/or his or her assistant surgeons to gain and ascertain a location of the forceps 34 of the surgical instrument 12 relative to the whole area of the body cavity 26. On the other hand, when selecting connection of the monitor 20 to the magnifying means 88, an magnified image of a real affected part of the abdominal cavity 26 near the forceps 23 of the surgical instrument 12 is displayed and examined on the monitor 20.

As just described above, according to the body cavity diagnostic system 10A thus constructed, the body cavity diagnostic unit 16A fixedly put in an incision 43 formed in the abdominal wall 42 captures an optical image of the interior of the body cavity 26 through the image pickup optical system 60 and signal transmits image signals corresponding to the optical image to the image processing unit 18 incorporated to the monitor 20 through the transmitter 62. In consequence, an assistant surgeon who conventionally operates the body cavity diagnostic unit 16A of the body cavity diagnostic system 10A of the present invention is made redundant Furthermore, the body cavity diagnostic unit 16A excites LEDs 52 with he built-in button type battery 54 and signal transmits image signal corresponding to an image of the interior of body cavity, it is not necessary to employ any light guide cable such as an optical fiber bundle for illumination of the interior of body cavity and any transmission cables between the image processing unit 18. In consequence, the number of assistant surgeons necessary for clinical treatment can be reduced as small as possibly without being accompanied by any inconvenience and a tidy environment is provided around the surgical bed 22 due to a simplified wiring system.

Furthermore, with this configuration, the body cavity diagnostic unit 16A including the LEDs 52 as an illumination light source realizes power saving, weight saving, miniaturization and overall compactness more effectively as compared with a conventional light source using an electric light bulb such as a small, low-power metal halide discharge lamp. In consequence, it is possible to form only a small incision in an abdominal wall of a patient for insertion of the body cavity diagnostic unit 16A. LEDs 52 produces a smaller heat release value as compared with the electric light bulb, the body cavity diagnostic unit 16A prevents body cavities from suffering thermal stimulations or burn injuries. Although LED 52 is not bounded by emission color, nevertheless, it is essential to employ the position representation mark M and LED 52 visually distinctive from each other. For example, when employing LED 52 having a red emission color, it is preferred to color the position representation mark M blue.

FIG. 4 shows a visual axis tracking system 100 as an alternative to the marker tracking means 90 for the image area selection and magnification means. As shown, the visual axis tracking system 100 comprising an infrared camera 102, namely an eye-monitor camera, and a regard point detector 104. The infrared camera 102 captures an image of an eye or eyes of an operating surgeon 28 and perceives a reflection point on the cornea with an infrared ray, preferably a highly rectilinear feeble laser ray. The regard point detector 104 finds a visual axis on the basis of information on the reflection point on the cornea and detects a point of regard on the image displayed on the monitor 20 on which the operating surgeon presently keeps observation in consideration of a distance between the operating surgeon 20 and the screen of the monitor 20. After detection of the point of regard on the image, the trimming means 86 trims the image so as to center the point of regard in the predetermined size of frame, and the magnifying means 88 magnifies the specific size of image at a desired magnification so as thereby to fill up a screen of the monitor 20. In this way, according to the visual axis tracking system 100, the operating surgeon and his or her assistant surgeons can examine an magnified image of a real affected part of the abdominal cavity 26 without using the forceps 23 of the surgical instrument 12 that has no position representation mark M thereon the forceps 23.

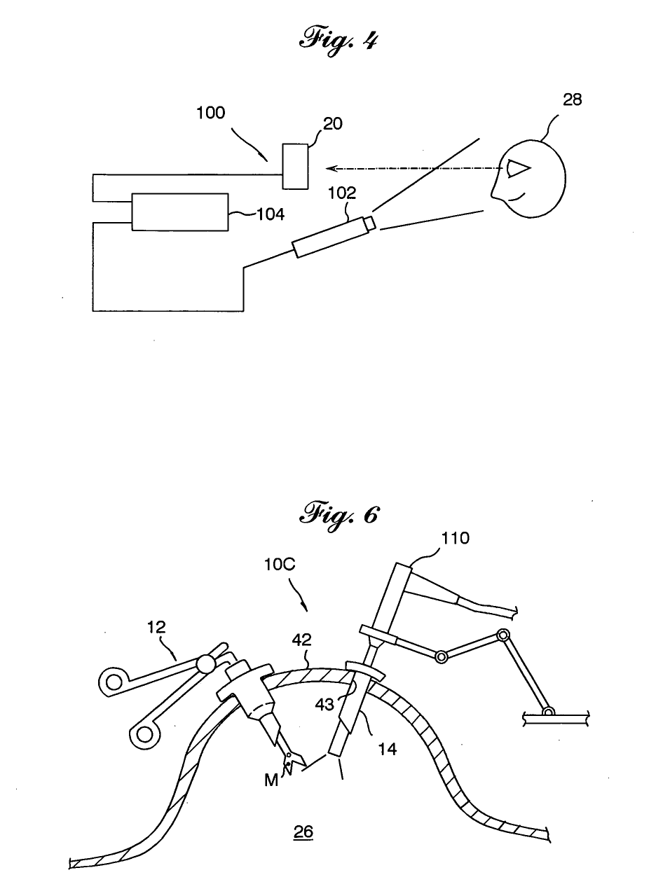

FIG. 5 shows a body cavity diagnostic system 10B according to an alternate embodiment of the present invention. The body cavity diagnostic system 10B is similar in constitution to that of the previous embodiment except for a body cavity diagnostic unit 16B provided with an interchangeable image pickup optical system mounted in a cylindrical unit housing 48B. The interchangeable image pickup optical system comprising an optical lens system 56 mounted within a cylindrical or tubular barrel 161 detachably received in a cylindrical opening 149 of the cylindrical unit housing 48B and a charge coupled device (CCD) image sensor 58 disposed behind the optical lens system 56. Signal wires 102 of the CCD image sensor 58 extend in a bore of the cylindrical barrel 161 and are connected to a connector 104 embedded in the cylindrical barrel 161. The connector 104 is electrically coupled to a counter connector 110 embedded in the cylindrical unit housing 48B when the cylindrical barrel 161 is received within the cylindrical opening 149 of the cylindrical unit housing 48B. This connector 110 is electrically connected to a base board 112 so as to transmit image signals from the CCD image sensor 58 to an image signal processor 74 (see FIG. 3) packaged on the base board 112. Since the remaining constituent parts are identical in structure and operation with those of the previous embodiment, a description of them is omitted.

According to the body cavity diagnostic system 10B having the body cavity diagnostic unit 16B used together with various interchangeable image pickup optical systems, the body cavity can be viewed with desired fields of view according to various interchangeable image pickup optical systems prepared for the system.

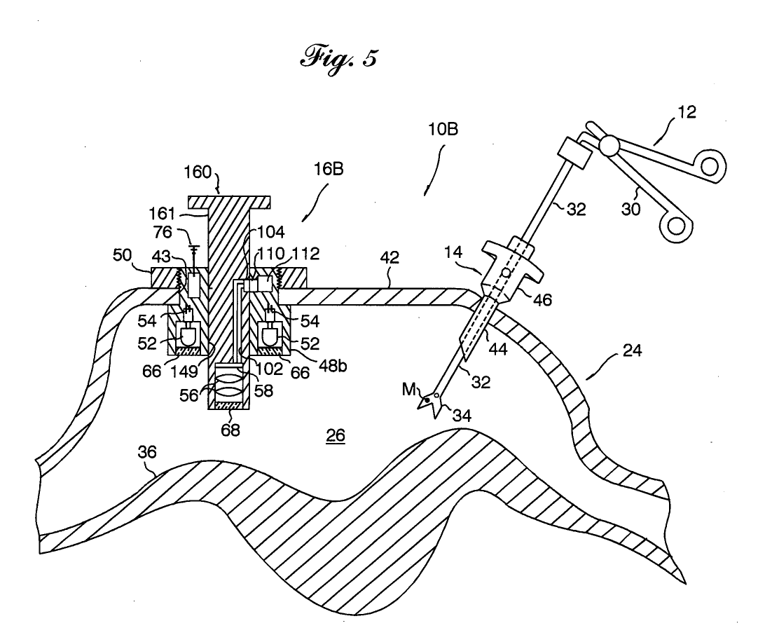

FIG. 6 shows a body cavity diagnostic system, more specifically a laparoscopic diagnostic and procedure system 10C, according to a further alternate embodiment of the present invention in which a combination of a laparoscope 110 and a hollow trocar 14 are used as the body cavity diagnostic unit 16A or 16B of the body cavity diagnostic system 10A or 10B according to the previous embodiment described above. An optical laparoscope, that is generally rigid, has traditionally been used in combination with a hollow trocar for insertion of the optical laparoscope into a body cavity. In order to change a line of sight with respect to the interior of the body cavity, it is usual practice to incline the rigid optical laparoscope within the hollow trocar or to incline the hollow trocar in an incision formed in a patient body. However, because there is only a small clearance between the rigid optical laparoscope and the hollow trocar and because the hollow trocar is strangled by the body wall, namely layers of skin and human flesh, of the patient, it is quite hard to train the laparoscope in a desired direction of view. This problem that the traditional rigid laparoscope encounters is easily overcome when using the laparoscopic diagnostic and procedure system 10C.

Although, the present invention has been described in association with the laparoscopic diagnostic and procedure system by way of example in the previous embodiments, nevertheless, it may be embodied in thoracoscopic diagnostic and procedure system as well as the laparoscopic diagnostic and procedure system. Further, various other embodiments and variants may occur to those skilled in the art which are within the scope and spirit of the invention, and such other embodiments and variants are intended to be covered by the following claims.

What is claimed is:

1. A body cavity diagnostic system comprising:
a medical procedure instrument for practicing a surgical procedure on an affected part within a body cavity of a patient; said medical procedure instrument being inserted into said body cavity through a cannula as an access way;
a body cavity diagnostic unit for optically capturing an image of an interior of said body cavity, said body cavity diagnostic unit being fixedly put in an incision formed in a body wall of said patient; and
a monitor unit for magnifying and displaying a desired part of said image on a monitor screen.

2. The body cavity diagnostic system as defined in claim 1, and further comprising a position representation mark formed on a distal end of at least one of said medical procedure instrument and said cannula, wherein said monitor unit trims said image so as to center said position representation mark in said desired area of said magnified image displayed on said monitor screen.

3. The body cavity diagnostic system as defined in claim 2, wherein said position representation mark is colored perceptibly differently from a body cavity color.

4. The body cavity diagnostic system as defined in claim 1, wherein said monitor unit comprises mark detection means for detecting said position representation mark, trimming means for trimming said image into a predetermined size of frame so as to center said position representation mark m said predetermined size of frame, and magnifying means for magnifying said trimmed image at a desired magnification.

5. A body cavity diagnostic system comprising:
a medical procedure instrument for practicing a laparoscopic surgical procedure on an affected part within a body cavity of a patient; said medical procedure instrument being inserted into said body cavity through a cannula as an access way;
a laparoscope having an optical system for optically capturing an image of an interior of said body cavity, said laparoscope being inserted into said body cavity through an incision formed in a body wall formed in a body wall of said patient; and
a monitor unit for magnifying and displaying a desired part of said image on a monitor screen.

6. The body cavity diagnostic system as defined in claim 5, and further comprising a position representation mark formed on a distal end of at least one of said medical procedure instrument and said cannula, wherein said monitor unit trims said image so as to center said position representation mark m said desired area of said magnified image displayed on said monitor screen.

7. The body cavity diagnostic system as defined in claim 6, wherein said position representation mark is colored perceptibly differently from a body cavity color.

8. The body cavity diagnostic system as defined in claim 5, wherein said monitor unit comprises mark detection means for detecting said position representation mark, timing means for trimming said image into a predetermined size of frame so as to center said position representation mark in said predetermined size of frame, and magnifying means for magnifying said trimmed image at a desired magnification.

9. A body cavity diagnostic system comprising:
a body cavity diagnostic unit for optically capturing an image of an interior of said body cavity and transmitting image signals of said image toward image receiving means for visual diagnosis, said body cavity diagnostic unit being fixedly put in an incision formed in a body wall of said patient;
wherein said body cavity diagnostic unit comprises illumination means for illuminating an interior of said body cavity, an image pickup optical system including solid state image sensing means for converting an image formed thereon into image signals, signal transmitting means for transmitting said image signals by air, and a power source for supplying electric power to said illumination means, said solid state image sensing means and said signal transmitting means.

10. The body cavity diagnostic system as defined in claim 9, wherein said image pickup optical system is interchangeable.

11. The body cavity diagnostic system as defined in claim 9, wherein said illumination means comprises light emitting diodes.

12. The body cavity diagnostic system as defined in claim 9, and further comprising image signal receiving means for receiving said image signals transmitted by air from said signal transmitting means, monitor means for displaying an visual image based on said image signals received by said signal receiving means, and a medical procedure instrument for practicing a surgical procedure on an affected part within said body cavity; said medical procedure instrument being inserted into said body cavity through a cannula as an access way.
